(19) United States
(12) Patent Application Publication
(10) Pub. No.: US 2020/0368675 A1
(43) Pub. Date: Nov. 26, 2020

(54) LOW ENERGY CONSUMPTION ANHYDROUS CO2 PHASE CHANGE ABSORPTION AGENT, AND REGENERATION METHOD AND APPLICATION THEREOF

(71) Applicant: Tiangong University, Tianjin (CN)

(72) Inventors: Qingsong ZHANG, Tianjin (CN); Xiaopeng LI, Tianjin (CN); Yunfei LI, Tianjin (CN); Juan ZHANG, Tianjin (CN); Yirong CAI, Tianjin (CN); Anan LI, Tianjin (CN); Xuedong JIN, Tianjin (CN); Pengfei LIU, Tianjin (CN); Li CHEN, Tianjin (CN)

(73) Assignee: Tiangong University, Tianjin (CN)

(21) Appl. No.: 16/959,436

(22) PCT Filed: Sep. 24, 2019
(86) PCT No.: PCT/CN2019/107412

§ 371 Date: Jul. 1, 2020

(30) Foreign Application Priority Data
Sep. 25, 2018 (CN) 201811103762.9

(51) Int. Cl. B01D 53/14; C07C 269/04

(52) U.S. Cl. CPC B01D 53/1425; B01D 53/1475; B01D 53/1493; B01D 2252/2041; B01D 2252/20421; B01D 2252/20431; B01D 2252/40; C07C 269/04

(57) ABSTRACT

Disclosed in the present disclosure are a low energy consumption anhydrous CO₂ phase change absorption agent, and a regeneration method and an application thereof, the absorption agent using a unitary diamine with a primary amine (NH₂—) and a tertiary amine (—N—), and not containing any other organic solvent, water, and ionic liquid; two alkyl branches are linked to a nitrogen atom of the tertiary amine, forming a certain hydrophobicity; after absorbing the CO₂, the diamine changes from a liquid phase to a solid phase, undergoing liquid-solid phase change to form white amino formate crystals.

### BACKGROUND

### Technical Field

The disclosure relates to the technical field of carbon dioxide capture, separation and recovery, in particular to a low energy consumption anhydrous CO₂ phase change absorbent and its regeneration method and application.

### Description of Related Art

Due to the potential impact on climate change, carbon dioxide (CO₂) emissions from coal-fired power plants and other industrial processes have attracted worldwide attention as the most important anthropogenic greenhouse gas (GHG). One of the environmental impacts of atmospheric CO₂ accumulation is global warming, which leads to climate problems such as polar melting, sea level rise and more severe weather patterns. It is generally accepted that CO₂ emissions from coal combustion need to be reduced immediately before more effective technologies or other renewable sources of energy can replace fossil fuels. At the 2009 World Climate Conference in Copenhagen, the Chinese government promised that the CO₂ emissions per unit of gross domestic product (GDP) by 2020 will be reduced by 40-45% compared with 2005, which posed a huge challenge to the reduction and control of CO₂ emissions in related industries in China. In the greenhouse gas improvement plan, carbon capture and storage (CCS) has been recognized as the key technology to reduce GHG emissions. Studies have found that CCS is the lowest cost technology to reduce climate change. For this reason, the widely accepted and mature method of CO₂ capture in industry is chemical absorption of amine aqueous solution. Amines commonly used for CO₂ removal include monoethanolamine (MEA), diethanolamine (DEA), triethanolamine (TEA) and other organic amines. However, the conventional amine aqueous solutions used for CO₂ capture has various disadvantages, such as equipment corrosion, solvent loss and large amount of heat used for solvent regeneration, and it takes a long time to reach equilibrium. Developing new absorbents with fast absorption rate, high absorption capacity and low regeneration energy consumption is the key to improve the separation and recovery process of CO_{2.}

Hasib-ur-Rahman [CO₂ Capture in Alkanolamine-RTIL Blends via carbamate Crystallization: Route to Efficient Regeneration[J]. Environ. Sci. Technol, 2012, 46, 11443-11450.] found that carbamate crystallization can be achieved by replacing the aqueous phase with a more stable and almost non-volatile room temperature ionic liquids based on imidazolium (RTIL); when CO₂ bubbles through the mixture diethanolamine/DEA-RTIL, 2-amino-2-methyl-1-propanol/AMP-RTIL respectively, the carbamate crystal salt product forms and migrates out of the liquid as a supernatant solid. This can greatly reduce the energy consumption of CO₂ regeneration. The decomposition temperature of DEA-carbamate (˜55° C.) is lower than that of AMP-carbamate (˜75° C.).

Cheng [Characterization of CO₂ Absorption and carbamate Precipitate in Phase-Change N-Methyl-1,3-diaminopropane/N,N-Dimethylformamide Solvent[J]. Energy Fuels 2017, 31, 13972-13978.]. A mixed solvent of N-methyl-1,3-diaminopropane (MAPA) and N,N-dimethylformamide (DMF) were prepared. In the MAPA/DMF solvent, MAPA-carbamate precipitate is formed after CO₂ is absorbed to reduce energy consumption of amine regeneration. Compared with MAPA/water solution (12.1 mg/g solvent), the CO₂ absorption of MAPA/DMF solvent (14.8 mg/g solvent) increased by 22%. For MAPA/DMF solvents, time is reduced by 22% to achieve CO₂ absorption equilibrium. The maximum CO₂ absorption rate of MAPA/DMF solvent is 30% higher than that of MAPA/water solution, because DMF shows higher CO₂ solubility and lower MAPA-carbamate solubility than water. DMF has certain toxicity and is not a friendly solvent.

CN1354036A of Nanhua Group Research Institute discloses a composite solvent of amine for recovering low partial pressure CO₂, which is characterized in that the solvent adopts a composite aqueous solution of monoethanolamine (MEA) and active amine, and the amine concentration is 1.5-7.5 mol/l, preferably 2.5-6 mol/l; Active amines are non-linear carbon chain alkanolamines with one or more steric hindrance effects on nitrogen atoms, which are characterized by an amine concentration of 2.5-6 mol/ml. The molar ratio of monoethanolamine to active amine is 1.95-4.65:1. Compared with traditional MEA solvent, the absorption capacity is improved by 40% and the energy consumption is reduced by 30%. Because the reaction mechanism of active amine with CO₂ is different from that of MEA, the absorption capacity of solution is increased and the energy consumption of regeneration is reduced. At the same time, active amine inhibits the impurities such as amino formaldehyde, aminoacetic acid, glyoxylic acid, oxalic acid, oxazolidinone, 1-(2-hydroxyethyl)-Imidazolinone and N-(2-hydroxyethyl)-ethylenediamine formed by degradation of MEA with O₂, CO₂, sulfide, and solves the problems of amine loss and equipment corrosion caused by degradation products.

CN104645782B of Shanghai Boiler Works Co., Ltd. discloses a CO₂ absorbent for post-combustion capture, which is characterized in that it includes a main absorbent which is polyethyleneimine (PEI), an auxiliary absorber which is one or more of tetraethylene pentamine (TEPA), monoethanolamine (MEA), N-methyl diethanolamine (MDEA), diethanolamine (DEA) and piperazine (PZ), antioxidant, corrosion inhibitor and water. Both components of main absorbents and auxiliary absorbents were organic amine compounds. The mass fraction of each component of the absorbent is :5-45% of the main absorbent, 5-30% of the auxiliary absorbent, 0.02-0.1% of the antioxidant, 0.02-0.1% of the corrosion inhibitor, and the rest is water. The total mass fraction of organic amines is 35-50%. The main characteristics of the disclosure are as follows: the main absorbent molecule has three amine groups of primary amine, secondary amine and tertiary amine at the same time, and has higher amine density compared with other types of organic amines. Therefore, the composite absorption solution has higher CO₂ absorption capacity and ensures faster absorption rate; the presence of a large number of tertiary amines leads to low reaction heat and reduces regeneration energy consumption. The composite solution has higher stability, and is matched with antioxidants and corrosion inhibitors to reduce solution loss in the circulation process.

CN101091864A of Dalian University of Technology has disclosed a new type of composite decarbonization solution, which is composed of main absorption component, auxiliary absorption component, activation component, corrosion inhibitor, antioxidant and water. Among them, the main absorption component is hydroxyethylenediamine (AEE), and the assistant absorption component includes 2-amino-2-methyl-1-propanol (AMP), N-methyl diethanolamine (MDEA) and triethanolamine (TEA), which can be used alone or mixed, but the total content of the assistant absorption component is 5-30% (mass fraction). Adding auxiliary absorption components can reduce the desorption temperature and make up for the deficiency of main absorption components. Among them, the active components include monoethanolamine (MEA), diethanolamine (DEA) and piperazine (PZ), which can be used alone or mixed, but the total content of the active components is 1-10% (mass fraction). The activated component mainly plays the role of activating the absorption assisting component, so that the absorption assisting component quickly reaches absorption saturation. The total amount of amine in the decarbonization solution in this disclosure is 35-55% (mass fraction). The corrosion inhibitors is sodium aluminate, and the antioxidants are sodium sulfite and copper acetate. The decarbonization solution in this disclosure has the advantages of large absorption capacity at 60-80 Nm³/m³, high desorption capacity at 45-55 Nm³/m³ and low desorption temperature.

In summary, according to the literature and patents, most of the chemical absorbents reported at present are composed of primary and secondary amines with fast absorption rate and tertiary amines with large absorption capacity, combined with water, N,N-dimethylformamide (DMF), ethanol and ionic liquids as auxiliaries, antioxidants and corrosion inhibitors. These absorbents have been greatly improved compared with the previous one-component absorbents, but there are still many problems in terms of absorption capacity, absorption rate and energy consumption, such as: complex separation process, high cost of ionic liquids, high viscosity, complex regeneration process, high energy consumption and toxicity, so it is difficult to be widely used in industrial processes. Therefore, it is necessary to provide a CO₂ absorbent with simple composition and low energy consumption, which has phase transition capability after absorbing CO₂, changes from liquid phase to solid phase, reducing the separation process, and has both absorption rate and absorption capacity, so as to optimize the process to meet its industrial application.

### SUMMARY

The technical problem to be solved by the present disclosure is to provide a low energy consumption anhydrous CO₂ phase change absorbent and its regeneration method and application. It does not contain water and other auxiliaries, has fast absorption rate, large absorption capacity, changes from liquid to solid after absorbing CO₂, and needs low regeneration temperature. Compared with other phase change absorbents, it reduces the separation process of rich phase and poor phase of CO₂ and the latent heat of solvents. Therefore, it can effectively reduce energy consumption and has a wide application prospect in the field of CO₂ absorption and separation in industry.

The absorbent of the disclosure adopts a single diamine compound with primary amine (NH₂—) and tertiary amine (—N—) at a concentration of 100%, and does not contain any other organic solvents, water or ionic liquid, wherein two alkyl branched chains are linked to the nitrogen atom of the tertiary amine to form certain hydrophobicity, and its molecular structure formula is shown in formula I:

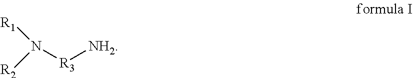

Among them, R₁, R₂ and R₃ are alkyl chains, and their typical representatives are as follows:

N,N-Dimethylaminoethylamine (DMEDA), R₁, R₂ are CH₃—, R₃ are —CH₂-CH₂—, using any one of:

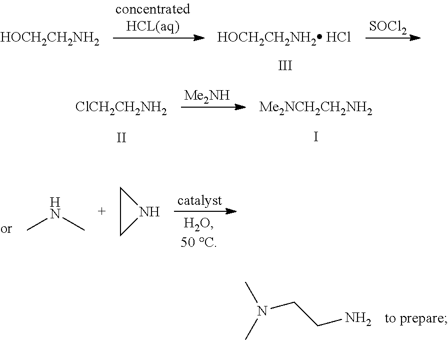

N,N′-Diethylethylenediamine (DEEDA), R₁, R₂ are CH₃-CH₂—, R₃ are —CH₂-CH₂—, using

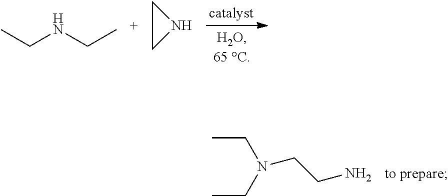

N,N′-Diisopropylethylenediamine (DIPEDA), R₁ and R₂ are CH₃—CH(CH₃)—, R₃ is —CH₂-CH₂—, using any one of:

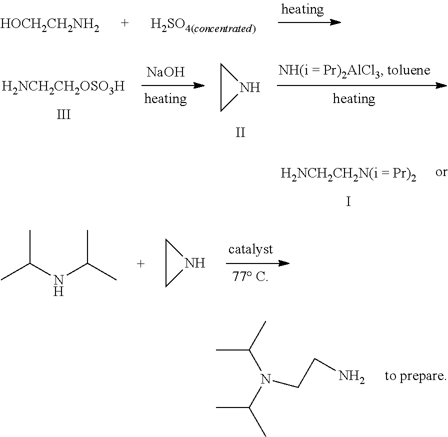

N,N′-Dibutylethylenediamine (DBEDA), R₁ and R₂ are CH₃-CH₂-CH₂-CH₂—, R₃ is —CH₂-CH₂—, using any one of:

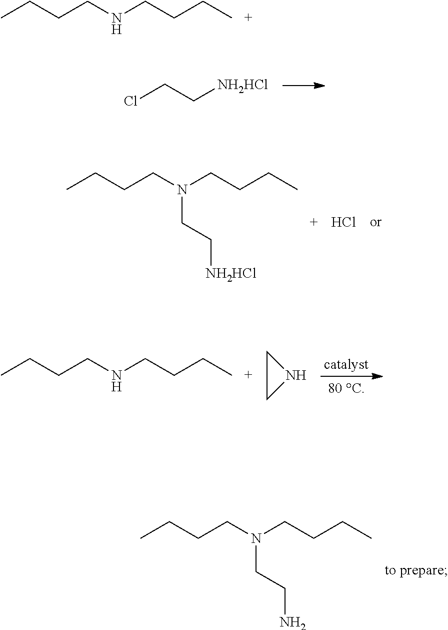

When the absorbent of the disclosure absorbs CO₂, the flow rate of CO₂ is 20-40 ml/min, the absorption saturation is achieved in 8-15 min, and the CO₂ load of the absorbent is 0.400-0.499 mol CO₂/mol amine.

The absorbent of the disclosure undergoes liquid-solid phase transformation after absorbing CO₂, and the solid white carbamate crystal is formed directly from the liquid phase. The decomposition temperature is 45-60° C., which is conducive to the regeneration of CO₂.

The mechanism of phase transition reaction of the absorbent of the disclosure is as follows:
R₁R₂NR₃NH₂+CO₂ [symbol] R₁R₂NR₃NH₂⁺COO⁻R₁R₂NR₃NH₂+R₁R₂NR₃NH₂⁺COO⁻ [symbol] R₁R₂NR₃NH₃⁺+R₁R₂NR₃NHCOO⁻.

The absorbent of the disclosure is an anhydrous single absorbent, and there is no excess liquid after absorbing CO₂, thus reducing the process of CO₂ enrichment phase separation and energy consumption.

The absorption load of the absorbent of the disclosure at 50° C. is higher than that of 30° C.-by 0.02 mol CO₂/mol amine.

The regeneration method of the absorbent after absorbing CO₂ is that the chemical reversible reaction occurs by heating, the CO₂, NH₂— is released from the carbamate solid decomposition to be regenerated, and the high purity CO₂ separated from the regeneration can be used subsequently, and the loss of phase change absorbent is low.

The disclosure provides a regeneration method of a low energy consumption anhydrous CO₂ phase change absorbent, which comprises the following steps:

1) Sealed sampling: take 2-4 g of the absorbent, the absorbent then absorbs CO₂ to transform into carbamate solid, and put it in a 20 ml glass reactor and seal it.

2) Phase change regeneration: put the glass reactor in the oil bath, control the oil bath temperature to 90-120° C., pass into N₂, the rate is 25-45 ml/min;

3) Regeneration calculation: heating the glass reactor for 40-80 minutes, taking it out, sealing it, weighing it, and calculating CO₂ emission and regeneration efficiency;

4) Phase change absorption: place the regenerated glass reactor contain diamine solution in a water bath of 20-50° C., introducing CO₂ at a rate of 20-40 ml/min;

5) Absorption calculation: take out the glass reactor after 40-60 min of phase change reaction, seal it, weigh it and calculate CO₂ absorption;

6) Cyclical implementation: repeat the above regeneration step and absorption step for 4 times to calculate the regeneration efficiency and CO₂ absorption capacity of diamine.

After four cycles of absorption and regeneration of the absorbent, the absorption efficiency of the absorbent is 70-85%.

The absorbent of the disclosure is applied to recovering CO₂ from chemical reaction tail gas, combustion flue gas and natural mixture gas, and removing CO₂ from urban gas and natural gas.

Compared with the background technology, the technical scheme has the following advantages:

The disclosure adopts a single organic solution containing primary amine (NH₂—) and tertiary amine (—N—), which is a transparent clarification solution before absorbing CO₂. After absorption, solid phase transformation occurs and solid crystalline salt products are formed. Compared with the traditional aqueous solution of organic amine, the absorption capacity is 0.400-0.499 mol CO₂/mol amine, the absorption capacity reaches saturation quickly in 8-15 minutes, and there is no liquid solvent, thus reducing the phase separation process. It can effectively reduce the latent heat of solvent in regeneration process and energy consumption, thus effectively overcome the shortcomings of traditional organic amine absorption method. It is a new type of economic and efficient CO₂ absorbent with practical application prospects, which is conducive to industrial promotion.

### BRIEF DESCRIPTION OF THE DRAWINGS

FIG. 1 shows the real-time appearance of CO₂ absorption by N,N-Dimethylaminoethylamine as absorbent.

FIG. 2 is a dynamic absorption process diagram of CO₂ absorption capacity and CO₂ absorption rate using N,N-Dimethylaminoethylamine as absorbent at 30-50° C.

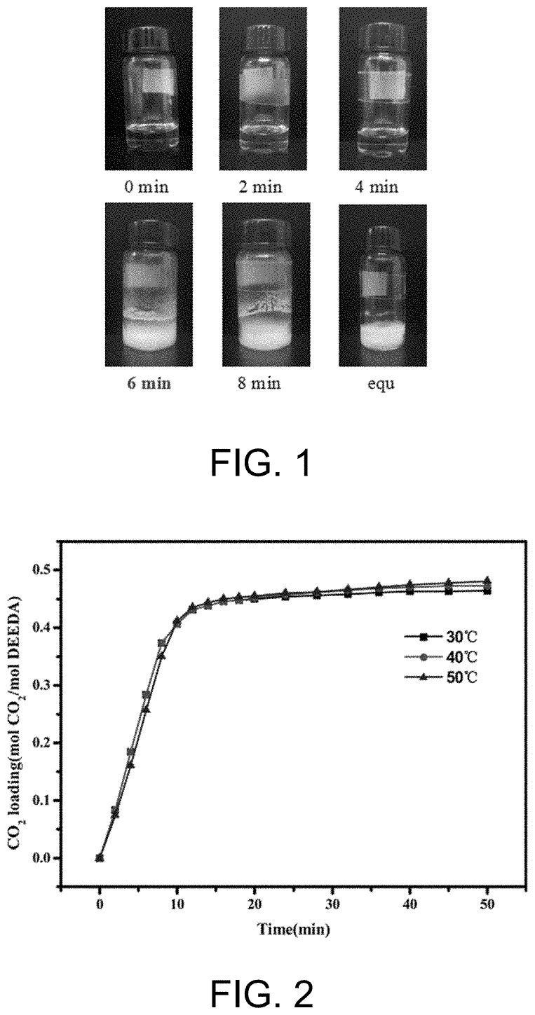

### DESCRIPTION OF THE EMBODIMENTS

The present disclosure is illustrated by the following examples, but the present disclosure is not limited to the following embodiments. The change implementation is included in the technical scope of the present disclosure without departing from the scope of purposes described before and after.

### EXAMPLE 1

The absorbent of the disclosure adopts a single diamine compound N,N′-diethyl ethylenediamine with both primary amine (NH₂—) and tertiary amine (N—) at a concentration of 100%, without any other organic solvent, water and ionic liquids, wherein the tertiary amine nitrogen atom is linked with two alkyl branchs to form a certain hydrophobicity and its molecular structure formula:

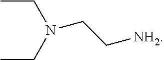

When the absorbent which is N,N′-diethyl ethylenediamine absorbs CO₂, the flow rate of CO₂ is 25 ml/min, and the absorption saturation is achieved in 10 minutes. The CO₂ loading of the absorbent is 0.465 mol CO₂/mol amine. The phase transformation reaction mechanism of the absorbent is as follows:

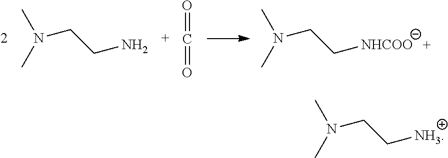

The disclosure provides a regeneration method of a low energy consumption anhydrous CO₂ phase change absorbent, which comprises the following steps:

1) Sealed sampling: take 2.80 g of the absorbent, the absorbent then absorbs CO₂ to transform into carbamate solid, and put it into a 20 ml glass reactor for sealing;

2) Phase change regeneration: put the glass reactor in the oil bath, control the temperature of the oil bath to 100° C., introduce N₂, and the rate is 30 ml/min;

3) Regeneration calculation: heating the glass reactor for 45 min, taking it out, sealing it, weighing it and calculating it, the CO₂ release amount is 0.370 mol CO₂/mol amine, and the regeneration efficiency is 79.57%;

4) Phase change absorption: place that regenerated glass reactor contain diamine solution in a 25° C. water bath, introducing CO₂ at a rate of 25 ml/min;

5) Absorption calculation: take out the glass reactor after 45 min of phase change reaction, seal it, weigh it, calculate it, CO₂ absorption is 0.368 mol CO₂/mol amine;

6) Cyclical implementation: repeat the above regeneration step and absorption step for 4 times, the regeneration efficiency of diamine is 77.20%, and the CO₂ absorption amount is 0.359 mol CO₂/mol.

The absorbent of the disclosure is applied to recover CO₂ from various chemical reaction tail gases.

### EXAMPLE 2

The absorbent of the disclosure adopts a single diamine compound N,N′-diethyl ethylenediamine with both primary amine (NH₂—) and tertiary amine (N—) at a concentration of 100%, without any other organic solvent, water and ionic liquids, wherein the tertiary amine nitrogen atom is linked with two alkyl branchs to form a certain hydrophobicity and its molecular structure formula:

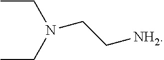

When the absorbent N,N′-diethyl ethylenediamine absorbs CO₂, the flow rate of CO₂ is 30 ml/min, and the absorption saturation is achieved in 12 minutes. The CO₂ loading of the absorbent is 0.464 mol CO₂/mol amine. The phase transformation reaction mechanism of the adsorbent is as follows:

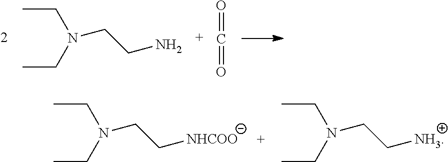

The disclosure provides a regeneration method of a low energy consumption anhydrous CO₂ phase change absorbent, which comprises the following steps:

1) Sealed sampling: Take 3.10 g of the absorbent, the absorbent then absorbs CO₂ to transform into carbamate solid, and put it into a 20 ml glass reactor for sealing;

2) Phase change regeneration: put the glass reactor in the oil bath, control the oil bath temperature to 105° C., pass into N₂, the rate is 35 ml/min;

3) Regeneration calculation: heating the glass reactor for 50 min, taking it out, sealing it, weighing it, and calculating it, the CO₂ release amount is 0.368 mol CO₂/mol amine, and the regeneration efficiency is 78.66%;

4) Phase change absorption: place the regenerated glass reactor containing the diamine solution in a 30° C. water bath and pass CO₂ at a rate of 30 ml/min;

5) Absorption calculation: take out the glass reactor after the phase change reaction for 50 min, seal it, weigh it, and calculate it. The CO₂ absorption capacity is 0.364 mol CO₂/mol amine;

6) Cyclical implementation: repeat the above regeneration and absorption steps four times, the regeneration efficiency of binary amine is 76.72%, and the CO₂ absorption amount is 0.356 mol CO₂/mol.

The absorbent of the disclosure is applied to recover CO₂ in combustion flue gas.

### EXAMPLE 3

The absorbent of the disclosure adopts a single diamine compound N,N′-diethyl ethylenediamine with both primary amine (NH₂—) and tertiary amine (N—) at a concentration of 100%, without any other organic solvent, water and ionic liquids, wherein the tertiary amine nitrogen atom is linked with two alkyl branchs to form a certain hydrophobicity and its molecular structure formula:

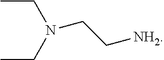

When the absorbent N,N′-diethyl ethylenediamine absorbs CO₂, the flow rate of CO₂ is 35 ml/min, and the absorption saturation is achieved in 9 minutes. The CO₂ loading of the absorbent is 0.413 mol CO₂/mol amine. The phase transformation reaction mechanism of the adsorbent is as follows:

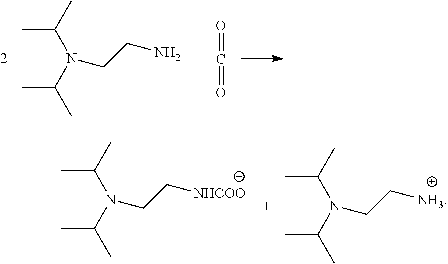

The disclosure provides a regeneration method of a low energy consumption anhydrous CO₂ phase change absorbent, which comprises the following steps:

1) Sealed sampling: take 3.30 g of the absorbent, the absorbent then absorbs CO₂ to transform into carbamate solid, and put it into a 20 ml glass reactor for sealing;

2) Phase change regeneration: put the glass reactor in the oil bath, control the oil bath temperature to 110° C., pass into N₂, the rate is 33 ml/min;

3) Regeneration calculation: heating the glass reactor for 55 min, taking it out, sealing it, weighing it, and calculating it, the CO₂ release amount is 0.341 mol CO₂/mol amine, and the regeneration efficiency is 82.57%;

4) Phase change absorption: Place the regenerated glass reactor containing the diamine solution in a 35° C. water bath and pass CO₂ at a rate of 35 ml/min;

5) Absorption calculation: take out the glass reactor after the phase change reaction for 55min, seal it, weigh it, and calculate it. The CO₂ absorption capacity is 0.339 mol CO₂/mol amine.

6) Cyclical implementation: repeat the above regeneration step and absorption step four times, the regeneration efficiency of binary amine is 80.39%, and the CO₂ absorption amount is 0.332 mol CO₂/mol.

The absorbent of the disclosure is applied to recover CO₂ in natural mixed gas.

### EXAMPLE 4

The absorbent of the disclosure adopts a single diamine compound N,N′ -diethyl ethylenediamine with both primary amine (NH₂—) and tertiary amine (N—) at a concentration of 100%, without any other organic solvent, water and ionic liquids, wherein the tertiary amine nitrogen atom is linked with two alkyl branchs to form a certain hydrophobicity and its molecular structure formula:

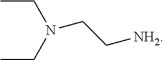

When the absorbent N,N′-diethyl ethylenediamine absorbs CO₂, the flow rate of CO₂ is 32 ml/min, and the absorption saturation is achieved in 13 minutes. The CO₂ loading of the absorbent is 0.493 mol CO₂/mol amine. The phase transformation reaction mechanism of the adsorbent is as follows:

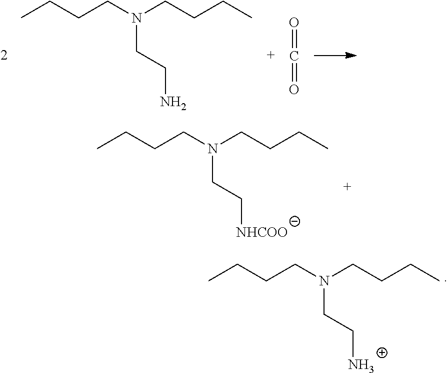

The disclosure provides a regeneration method of a low energy consumption anhydrous CO₂ phase change absorbent, which comprises the following steps:

1) Sealed sampling: take 3.50 g of the absorbent, the absorbent then absorbs CO₂ to transform into carbamate solid, and put it into a 20 ml glass reactor for sealing;

2) Phase change regeneration: put the glass reactor in the oil bath, control the oil bath temperature to 115° C., pass into N₂, the rate is 40 ml/min;

3) Regeneration calculation: heating the glass reactor for 60 min, taking it out, sealing, weighing, and calculating, the CO₂ release amount is 0.409 mol CO₂/mol amine, and the regeneration efficiency is 82.96%;

4) Phase change absorption: place the regenerated glass reactor containing the diamine solution in a 40° C. water bath and pass CO₂ at a rate of 32 ml/min;

5) Absorption calculation: take out the glass reactor after the phase change reaction for 52 min, seal it, weigh it, and calculate it. The CO₂ absorption capacity is 0.390 mol CO₂/mol amine.

6) Cyclical implementation: repeat the above regeneration step and absorption step four times, the regeneration efficiency of binary amine is 76.87%, and the CO₂ absorption amount is 0.379 mol CO₂/mol.

The absorbent of the disclosure is applied to remove CO₂ from urban gas, natural gas, etc.

According to the above examples, the new CO₂ phase change absorbent has the advantages of fast absorption rate, large absorption load, reduced phase separation process, high regeneration efficiency in a short time, water-free solvent, reduced latent heat of solvent, reduced energy consumption, and can be widely used to recover CO₂ from various chemical reaction tail gas, combustion flue gas and natural mixed gas, and can also be used to remove CO₂ from urban gas and natural gas.

1. A low-energy anhydrous CO₂ phase change absorbent, wherein the absorbent uses a single diamine compound having both primary amine (NH₂—) and tertiary amine (—N—) at a concentration of 100%, free of any other organic solvents, water and ionic liquids, wherein the tertiary amine nitrogen atom has two alkyl branches linked to it, which constitutes a certain degree of hydrophobicity, and its molecular structure formula is shown in Formula I: 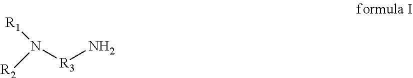
among them, R₁, R₂ and R₃ are alkyl chains, and their typical representatives are:
N,N′-dimethylenediamine (DMEDA), R₁ and R₂ are CH₃—, and R₃ is —CH₂-CH₂—,
N,N′-diethylenediamine (DEEDA), R₁ and R₂ are CH₃-CH₂—, and R₃ is —CH₂-CH₂—,
N,N′-diisopropylenediamine (DIPEDA), R₁ and R₂ are CH₃-CH (CH₃)—, and R₃ is —CH₂-CH₂—,
N,N′-n-butyl ethylenediamine (DBEDA), R₁ and R₂ are CH₃-CH₂-CH₂-CH₂—, and R₃ is —CH₂-CH₂—.

2. The CO₂ phase change absorbent according to claim 1, wherein the mechanism of phase change reaction is:
R₁R₂NR₃NH₂+CO₂ [symbol] R₁R₂NR₃NH₂⁺COO⁻R₁R₂NR₃NH₂+R₁R₂NR₃N ⁺COO⁻ [symbol] R₁R₂NR₃⁻+R₁R₂NR₃N COO⁻.

3. The CO₂ phase change absorbent according to claim 1, wherein when the absorbent absorbs CO?, the flow rate of CO₂ is 20-40 ml/min, the absorption saturation is achieved in 8-15 min, and the CO₂ loading of the absorbent is 0.400-0.499 mol CO₂/mol amine.

4. The CO₂ phase change absorbent according to claim 1, wherein the absorbent undergoes liquid-solid phase transformation after absorbing CO₂, and a solid phase white carbamate crystal is directly formed from a liquid phase with a decomposition temperature of 45-60° C., which is favorable for CO₂ regeneration.

5. The CO₂ phase change absorbent according to claim 1, wherein the absorbent is an anhydrous single absorbent, there is no excess liquid after adsorbing CO₂, which reduces the process of CO₂ enrichment phase separation and reduces energy consumption.

6. The CO₂ phase change absorbent according to claim 1, wherein the absorption load of the absorbent at 50° C. is higher than that at 30° C. by 0.01-0.02 mol CO₂/mol amine.

7. A regeneration method of a low energy anhydrous CO₂ phase change absorbent, including the following steps:
1) sealed sampling: take 2-4 g of the absorbent according to claim 1, the absorbent then absorbs CO₂ to transform into carbamate solid, and put it in a 20 ml glass reactor and seal it;
2) phase change regeneration: place the glass reactor in an oil bath, control the temperature of the oil bath to 90-120° C., pass N₂ at a rate of 25-45 ml/min;
3) regeneration calculation: heat the glass reactor for 40-80 min, take it out, seal it, weigh it, calculate the released amount of CO₂ and regeneration efficiency;
4) phase change absorption: place the regenerated glass reactor containing the diamine solution in a water bath at 20-50° C. and pass CO₂ at a rate of 20-40 ml/min;
5) absorption calculation: after 40-60 min of phase change reaction, take out the glass reactor, seal it, weigh it, and calculate the CO₂ absorption; and
6) cyclical implementation: repeat the regeneration step and absorption step for 4 times, and calculate the regeneration efficiency of diamine and the absorption capacity of CO₂.

8. The regeneration method of the low energy consumption anhydrous CO₂ phase change absorbent according to claim 7, wherein the regeneration method of the absorbent after absorbing CO₂ is a chemical reversible reaction through heating, CO₂ is released by decomposition of the carbamate solid, and NH₂— is regenerated, the regenerated and separated high purity CO₂ can be used subsequently, and the loss of phase change absorbent is low.

9. The regeneration method of the low energy consumption anhydrous CO₂ phase change absorbent according to claim 7, wherein during the four cycles of absorption and regeneration of the absorbent, the absorption time and the regeneration time are controlled to 40-80 minutes, and the absorption efficiency of the absorbent reaches 70-85%.

10. An application of a low energy consumption anhydrous CO₂ phase change absorbent according to claim 1, wherein the absorbent is applied to recover CO₂ from chemical reaction tail gas, combustion flue gas and natural gas mixture, and to remove CO₂ from urban gas and natural gas.
